(19) United States
(12) Patent Application Publication
(10) Pub. No.: US 2025/0311914 A1
(43) Pub. Date: Oct. 9, 2025

(54) Bed-Mounted Surgical Tool Organization System

(71) Applicant: University of Cincinnati, Cincinnati, OH (US)

(72) Inventors: Kathleen Meyer, Cincinnati, OH (US); Aseem Jain, Cincinnati, OH (US); Aaron Friedman, Cincinnati, OH (US); Nathan Van Damme, Cincinnati, OH (US)

(73) Assignee: University of Cincinnati, Cincinnati, OH (US)

(21) Appl. No.: 18/860,508

(22) PCT Filed: Apr. 25, 2023
(86) PCT No.: PCT/US23/19752

§ 371 Date: Oct. 25, 2024

Related U.S. Application Data
(60) Provisional application No. 63/334,474, filed on Apr. 25, 2022.

(51) Int. Cl. A61B 1/00; A61B 50/33

(52) U.S. Cl. CPC A61B 1/00149; A61B 50/33

(57) ABSTRACT

An apparatus for securing surgical instruments to make them accessible by a surgeon while performing an operation on a patient is provided. The apparatus includes an articulated positioning arm that has a first arm section and a second arm section. The apparatus also includes an instrument support surface extending from the second arm section of the articulated positioning arm. The apparatus further includes a mounting interface that is capable of mounting the apparatus on a surgical bed. In addition, the apparatus includes a second device support connected to the mounting interface.

### CROSS-REFERENCE TO RELATED APPLICATIONS

This application is a continuation of PCT Application No. PCT/US23/19752 filed Apr. 25, 2023, which claims the benefit of the filing date of U.S. Provisional Application Ser. No. 63/334,474, filed on Apr. 25, 2022, the disclosures of which are incorporated by reference herein in their entirety.

### TECHNICAL FIELD

The present invention relates to systems for organizing surgical tools.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Microlaryngoscopic surgery entails utilizing an array of tools to access the larynx (vocal cords) with microscopic visualization. In the current surgical paradigm, a scrub tech arranges tools on a Mayo stand and hands them to the surgeon. The handoff between scrub tech and surgeon creates the opportunity for use errors. Surgical tools can be mishandled, confused, or dropped during this handoff. The multiple tool exchanges and the communication required increase surgical time and therefore expense. Because microlaryngoscopy requires the surgeon to visualize the surgical field through microscope oculars, nearby access to tools is essential.

There is opportunity to reduce surgical errors and speed up procedure time by giving the surgeon direct control of the instrument stand. However, a typical Mayo stand is designed for a standing surgeon performing procedures in the thoracic or abdominal cavity. It does not have ideal ergonomics to be positioned near the patient head and the laryngologist. Microlaryngoscopic tools can have long distal end effectors, which increase the risk of extending beyond the border of a surgical tray in a manner that may lead to inadvertent contact or falling to the floor. Therefore, a need still exists for an improved organizing system to hold surgical instruments and to present them to a surgeon.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, an apparatus for securing surgical instruments to make them accessible by a surgeon while performing an operation on a patient is provided. The apparatus includes an articulated positioning arm that has a first arm section and a second arm section. The first arm section is oriented in a substantially horizontal position and the second arm section can be oriented in multiple positions. The apparatus also includes an instrument support surface extending from the second arm section of the articulated positioning arm. The instrument support surface can be oriented in multiple positions. The apparatus further includes a mounting interface that is capable of mounting the apparatus on a surgical bed. The mounting interface is connected to the articulated positioning arm. In addition, the apparatus includes a second device support connected to the mounting interface. The second device support is configured to support one or more surgical tools.

In one embodiment, the first arm section of the articulated positioning arm and the second arm section of the articulated positioning arm are connected at a perpendicular angle. In another embodiment, the instrument support surface comprises at least one removeable tool support. In one embodiment, the instrument support surface is configured to support endoscopic surgical tools. In another embodiment, the instrument support surface further comprises a surgical instrument tray. In this embodiment, the surgical instrument tray includes features that align and secure surgical instruments.

In one embodiment, the instrument support surface is adjustable. In another embodiment, the instrument support surface can be positioned at an angle of from about 5 degrees to about 80 degrees from horizontal. In one embodiment, the instrument support surface can be positioned at an angle of from about 10 degrees to about 45 degrees from horizontal. In another embodiment, the second device support is configured to support one or more endoscopic surgical tools. In one embodiment, the second arm section of the articulated positioning arm comprises at least two ball joints.

In another embodiment, the surgical instruments are endoscopic instruments and the second device support is an endoscopic tool holder. In one embodiment, the endoscopic instruments comprise microlaryngoscopic instruments. In another embodiment, the apparatus further includes control surfaces that facilitate repositioning of the instrument support surface while maintaining sterility. In one embodiment, the instrument support surface further comprises flexible negative features that are capable of capturing distal portions of surgical instruments. In another embodiment, the instrument support surface further includes features that extend from the surgical instrument tray which are capable of aligning surgical instruments and arresting any undesired lateral motion. In one embodiment, the instrument support surface further comprises one or more flexible protrusions that are capable of supporting the surgical instruments.

In another embodiment, the apparatus is capable of being sterilized via autoclave. In one embodiment, the apparatus is capable of being sterilized using ethylene oxide. In another embodiment, the apparatus is capable of being sterilized via gamma radiation. In one embodiment, the surgical instruments are neurosurgical instruments. In another embodiment, the surgical instruments are ophthalmic instruments. In one embodiment, the surgical instruments are laparoscopic instruments. In another embodiment, the surgical instruments are otolaryngologic instruments. In one embodiment, the articulated positioning arm further comprises bend-and-stay arms, counterbalancing arms or combinations thereof. In one embodiment, the apparatus uses mechanical latches or magnets to secure and align instruments. In one embodiment, the instrument support surface can extend and retract to accommodate for surgical instruments of different sizes. In one embodiment, the instrument support surface can split into modular components based on surgical need.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1A is a schematic of an embodiment of the surgical tool organization system of the present invention.

FIG. 1B is a schematic of an embodiment of subassembly 1 of the present invention.

FIG. 1C is a schematic of an embodiment of subassembly 2 of the present invention.

FIG. 1D is a schematic of an embodiment of subassembly 3 of the present invention.

FIG. 1E is a schematic of an embodiment of subassembly 4 of the present invention.

FIG. 2 is a schematic of a perspective view of subassembly 1, the endoscopic tray holder system.

FIG. 3 is a schematic of silicone inserts used to secure endoscopic tools as part of subassembly 1 according to the present invention.

FIG. 4 is a schematic of a perspective view of subassembly 4, the endoscopic telescope holder and subassembly 3.

FIG. 5A is a box and whisker plot showing instrument pass times (IPT) for overall control vs device (a holder according to the present invention) cases. Individual circles represent outlier data points (>3 standard deviations from the mean); n represents the number of instrument passes.

FIG. 5B is a box and whisker plot showing stratified data by type for instrument pick up (IPU) and instrument return (IR). Individual circles represent outlier data points (>3 standard deviations from the mean); n represents the number of instrument passes.

FIG. 6 is a graph showing instrument drops and communication errors in control cases versus cases using a holder according to the present invention.

FIG. 7A is a box and whisker plot showing instrument pass times (IPT) in control vs inventive holder cases as stratified by attending surgeon-rated scrub technician (ST) performance. n represents the number of instrument passes.

FIG. 7B is box and whisker plot showing instrument pass times (IPT) in control vs inventive holder cases as stratified by ST self-rated familiarity with procedure. n represents the number of instrument passes.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, “articulated” means having two or more sections connected by a flexible joint.

As used herein, “Instrument Pass Time” (IPT) means the time from when a surgeon calls for an instrument to when he or she grasps it.

As used herein “IPU” means instrument pick up.

As used herein “IQR” means inter-quartile range.

As used herein “IR” means instrument return.

As used herein “ST” means surgical technician.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one”.

The shortcomings of current surgical organizing systems can be addressed by a system that presents tools to the surgeon at an angle for better visualization and access. However, current surgical trays cannot angle toward the surgeon, and have no features to prevent tools from rolling to the ground if they were angled. The present invention provides a solution to this problem. In one embodiment, it involves a novel system for presenting tools to a surgeon during microlaryngoscopic surgery.

The surgical tool holder of the present invention has the potential to improve operative efficiency. This device, in addition to decreasing the amount of time spent passing instruments, leads to a more consistent instrument handoff experience, and reduces the number of communication errors involved in handling of surgical tools such as endoscopic laryngeal and airway microsurgery (ELAM) instruments without increasing instrument drop rate. As shown in the Examples, use of the surgical tool holder of the present invention resulted in a 1.3 second mean decrease in Instrument Pass Time (IPT). While this may seem insignificant individually, extrapolated over time this could save valuable OR resources, particularly for longer or higher pass per unit time cases.

As discussed below, data was collected from 25 device cases and 23 control cases among three different laryngologists. Average IPT was nearly three times quicker for the device (0.80 seconds, n=1175 passes) compared to controls (2.09 seconds, n=1208 passes) [p<0.001]. IPT interquartile range was five times higher for control (1.65 seconds) versus device cases (0.42 seconds). Instrument drop rate (IDR) was not significantly different [p=0.48]; however, device cases had significantly lower communication errors compared to control cases [p=0.01]. Surgeons and surgical assistants were similarly satisfied with the device on a 5-point Likert scale (mean: 4.2/5, standard deviation: 0.92). This data shows that the surgical instrument holder of the present invention can improve ELAM operative workflow by reducing instrument passing time and variability without increasing IDR.

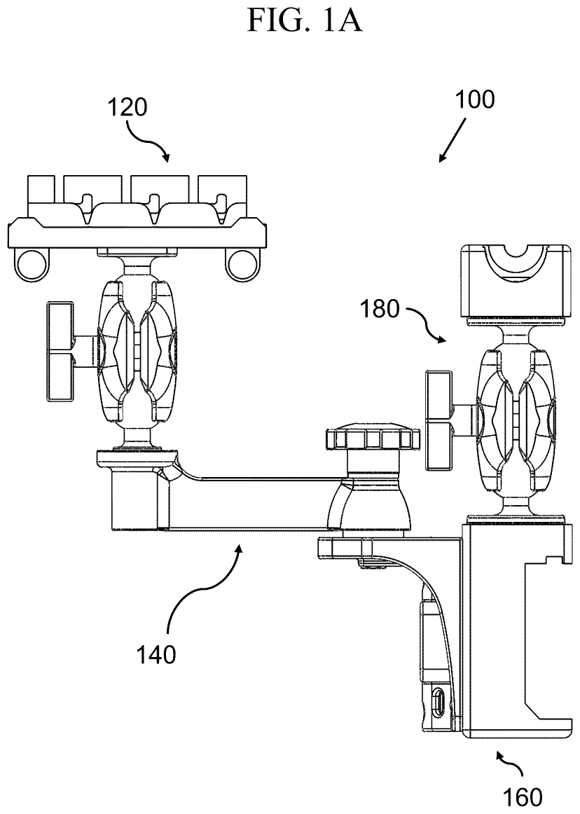

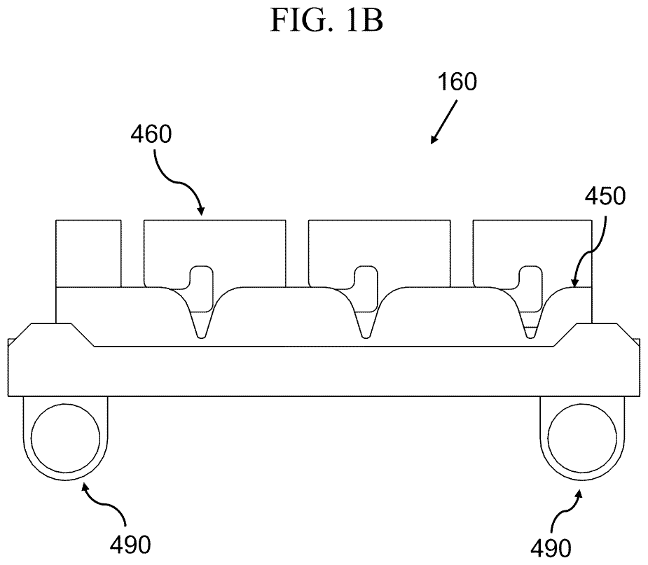

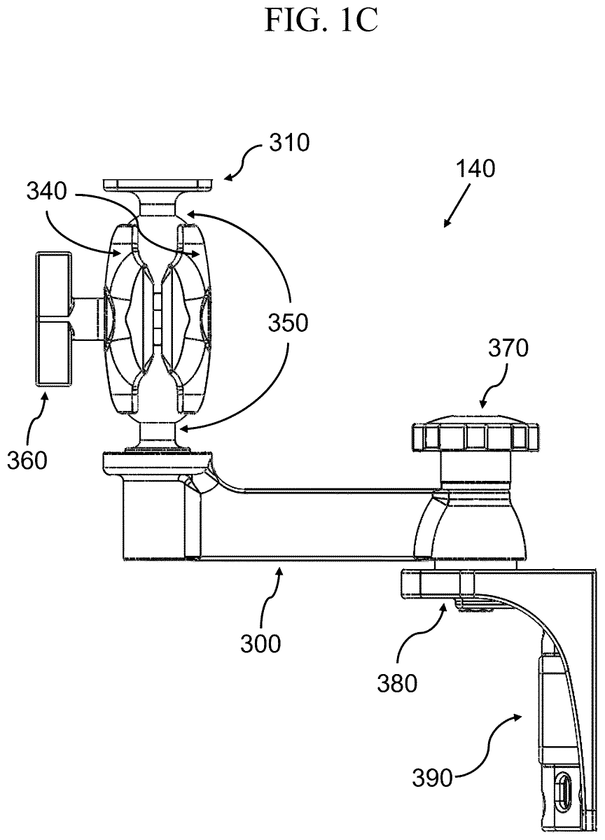

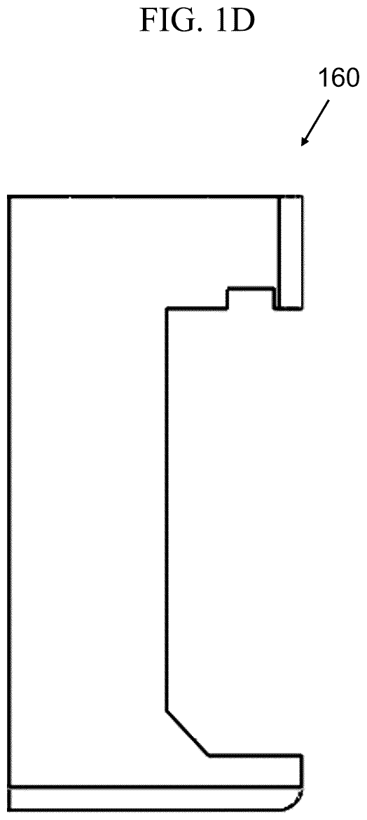

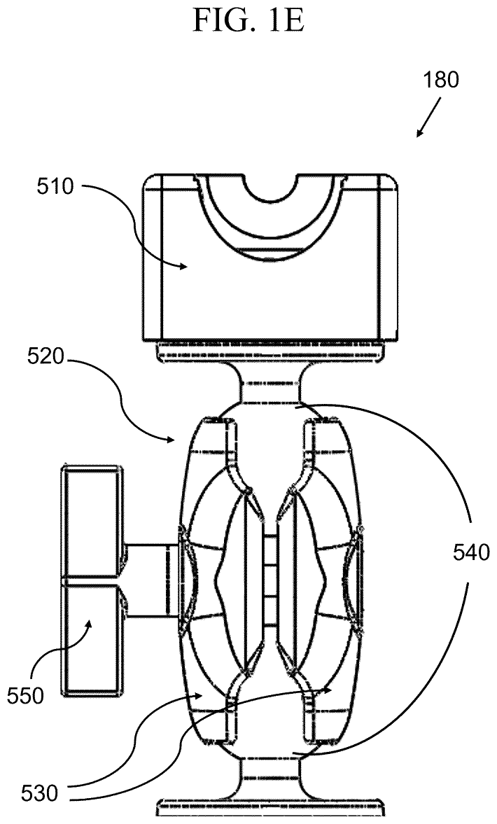

Referring to FIG. 1A, a broad overview of the surgical tool organization system 100 of the present invention is presented. This particular embodiment is designed to support endoscopic instruments. It is referred to as an endoscopic tool organization system (ETOS). This embodiment of the system comprises four subassemblies, Subassembly 1 (SA1) 120, Subassembly 2 (SA2) 140, Subassembly 3 (SA3) 160 and Subassembly 4 (SA4) 180.

FIG. 1B is an isolated view of SA1 120. SA1 consists of an instrument support surface further described below. FIG. 1C an isolated view of SA2 140. SA2 functions as an articulated positioning arm system for SA1 120. In the embodiment shown in FIG. 1C, SA2 140 is an articulated positioning arm including a first arm section 300 that allows for pivoting around a mounting location 380 on an attachment bracket 390. The attachment bracket 390 connects to SA4 180. The location of the first arm section 300 can be fixed in place by tightening the screw knob 370. SA2 140 also includes a second arm section 310. In alternative embodiments, SA2 incorporates other generic positioning arm systems. Examples of alternate positioning arm systems include bend-and-stay arms and counterbalancing arms. In this embodiment, second arm section 310 comprises two metal positioning arm segments 340 and three rubber pivot points (not shown). This embodiment allows for manipulation and adjustment, but an alternative embodiment with more or fewer pivot points/arms are possible. Ball joints 350 are tightened by the tension knob 360 which advances a screw mechanism (not shown) that pulls the arm segments 340 together and therefore increases force applied to the ball joints 350 holding it in place.

FIG. 1D is an isolated view of SA3 160, which is a mounting interface for all the subassemblies to be mounted on an operating room (OR) bed (not shown). In this embodiment, the mounting interface utilizes a screw clamp (not shown) to secure the ETOS to the OR bed. Other embodiments may include other mechanisms to mount the ETOS to bed.

FIG. 1E is an isolated view of SA4 180, which is a second device support that functions as a surgical tool holder. It is further discussed below.

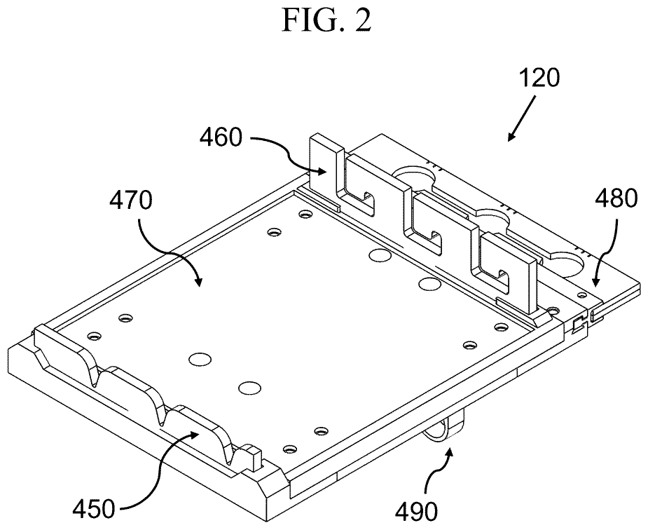

Referring to FIG. 2, a schematic of SA1 120, the instrument support surface, is shown. In this embodiment, the instrument support surface consists of two silicone inserts 450, 460 that are used to hold endoscopic tools mounted on a plastic custom 3D printed tray. In an alternate embodiment, the tray comprises metal such as aluminum. The distal end of an endoscopic tool can be placed in the removeable tool support 460, while the proximal end of the tool is mounted in removeable tool support 450. In one embodiment, the removeable tool supports are made of silicone. Other materials may be used. The materials may either be capable of sterilization or are disposable.

In one embodiment, the tray bed 470 is used to hold a silicone mat (not shown) that also assists with securing the surgical tools. In this embodiment, the instrument support surface 120 is adjustable. In one embodiment, the instrument support surface 120 can be positioned at an angle of from about 5 degrees to about 80 degrees from horizontal. In another embodiment, the instrument support surface 120 can be positioned at an angle of from about 10 degrees to about 45 degrees from horizontal. Additionally, this system has loops 490 placed underneath the tray that allows for passing of tubing that prevents tubing from interfering with placing and picking up surgical tools. The distal end of the instrument support surface has a modular component 480 that can be used for various attachments to the device. For example, a slot design with a pin joint can be used to attach other components. In one embodiment, this allows attaching a tray extender. In this current embodiment, the attachment is a platform to hold small items used in surgery such as gauze pads, plegets, epiplegets, or similar items. Other embodiments may allow for different attachments to be mounted onto the surgical tray.

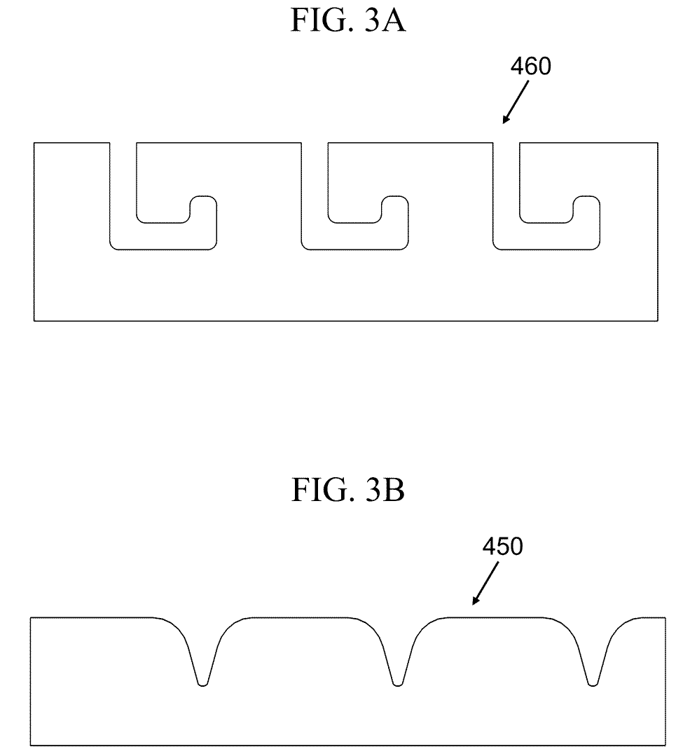

FIG. 3 is an isolated view of removeable tool supports 450 and 460 that, in this embodiment, are used to secure endoscopic tools. The tool supports may be removed from the instrument support surface for cleaning, or to replace with a tool support designed for another type of surgical instrument. Other embodiments may use different insert designs for securing instruments.

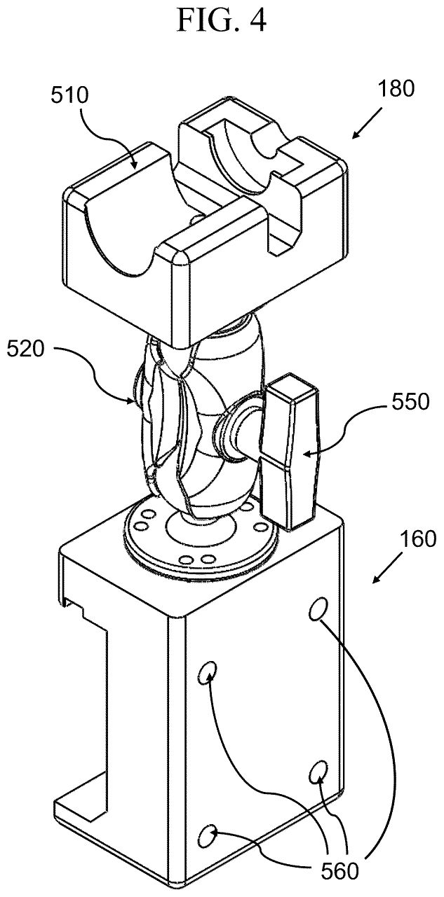

FIG. 4 provides a detailed illustration of SA4 180, the second device support. In this embodiment, the second device support 180 functions as an endoscopic telescopic holder. The endoscopic telescope is a crucial component of laryngeal procedures, enabling users to capture a view of the airway to ensure proper ventilation. Unfortunately, this tool is cumbersome and can interfere with the passing of instruments. This embodiment of SA4 180 is mounted to SA3 160 and SA3 160 has apertures 560 that allows SA3 160 and SA4 180 to be mounted onto the other subassemblies. In this embodiment, SA4 180 consists of a tool cradle 510 for holding an endoscopic telescope. The tool cradle 510 is connected to a positioning arm 520 that enables surgeons to position the second device support. Positioning arm 520 operates in a similar manner to the positioning mechanism of SA2 140. Referring to FIG. 1E, which is an alternate view of SA4 180, the positioning arm 520 includes positioning arm segments 530, ball joints 540 and a tension knob 550.

Some embodiments of the present invention may be sterilized before and/or after a procedure. One such method of sterilization could be steam via an autoclave. In one embodiment, the design uses autoclaved removeable tool supports 450 and 460 and 3D printed plastic components that are sterilized with alcohol or other disinfectants. The sterilized organizer may then be stored in the sterile container until the next required use. In one embodiment, this sterilization occurs with the tools still inside the organizer. In another embodiment, the organizer is sterilized using another technique known in the art, including but not limited to ethylene oxide, or gamma radiation.

While the embodiment described above would be reusable following re-sterilization, another embodiment could be manufactured for single-use. Those skilled in the art can appreciate that removing the requirement of re-sterilization could facilitate a lighter, less expensive system. Another embodiment of the system has a reusable portion encompassing the adjustable arm and mount that mates with a single-use organizer that is provided in a sterile package.

While this specification describes an embodiment describing the application of the invention to microlaryngoscopic surgery, those skilled in the art will appreciate how this invention can apply to other surgical specialties, including but not limited to ophthalmic surgery and neurosurgery.

### Experimental Data

The data presented in Examples 1 and 2 supports the conclusion that using the endoscopic tool holder of the present invention leads to a lower IPT and inter-quartile range (IQR). The decrease in IQR suggests that using this device translates to less variability in instrument pass time. This conclusion is further supported by an overall decrease in the number and magnitude of outliers between control cases and device cases. This decrease in IPT variability could be attributed to having preloaded appropriate instruments in the device as well as having fewer interactions with STs.

The relationship between IPU and IR in control cases (IPU time>IR time) compared to device cases (IPU time<IR time) can also be potentially explained as one of the benefits of having preloaded instruments in the device. During control cases, the surgeon would ask for an instrument, prompting the ST to select the appropriate one and hand it to the surgeon; however, during instrument returns, the surgeon could place his or her hand out and ask the ST to take it-a considerably quicker task. For device cases, since the instruments were preloaded in the holder, the surgeon was able to pick up instruments quickly; however, returning an instrument, depending on the surgeon's skill with using the device, would take slightly longer, since it involved manipulating the tool to fit back in the device. Despite this, overall IPT was significantly lower while using the device for both IPU and IR.

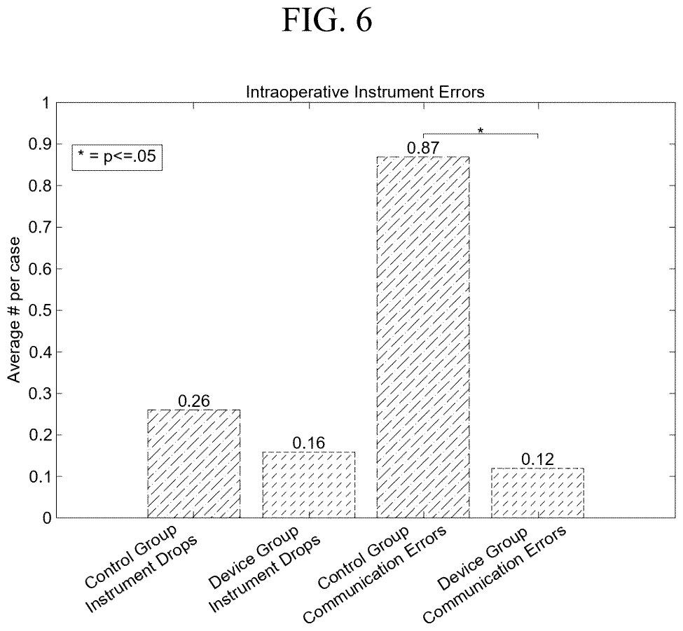

An important aspect for the holder design of the present invention was to ensure that the device could securely hold the endoscopic instruments without making it difficult for the surgeon to pick up and return them. The data presented in FIG. 6 support that this goal is achieved by the present invention. Communication errors were, however, significantly lower in device cases. Anecdotally, the most common communication error in control cases was a ST initially picking up an incorrect instrument. During device cases, preloading instruments decreased this issue.

One skilled in the art may consider an alternative to the holder, which would be to use Mayo stands on either side of the patient's head. These stands could allow a surgeon to self-serve frequently used instruments. However, the size of these stands is much larger than the developed holder trays, and real estate in the vicinity of the patient's head is already occupied by other equipment (eg surgical microscope, laser) and/or personnel (ST, anesthesiologist, laser technician). Additionally, bed mounted holders easily move with repositioning of the OR bed, which is often necessary intra-operatively; Mayo stands would need to be separately raised/lowered with each bed reposition. Moreover, the holder offers intrinsic organization of instruments for easy surgical visibility/access that would not be inherent to simply laying them on a Mayo stand. Finally, one of the most frequently utilized instruments in ELAM is a microlaryngeal suction, and the attached suction tubing typically has “memory” that can cause the suction itself or other unrestrained instruments to easily fall off a Mayo stand. The holder of the present invention not only keeps suctions more secure at the instrument end, but also includes built in suction tubing loops 490 to minimize this chance it of falling out.

### EXAMPLES

### Example 1

Institutional Review Board approval was obtained to study the holder of the present invention during ELAM cases that were classified as phonosurgery, neoplastic (laryngotracheal papillomatosis, glottic dysplasia, or early glottic carcinoma), or airway (laryngotracheal) stenosis. Operations involving flexible bronchoscopy or pharyngoesophageal procedures were not included since the holder was not designed for these surgical tools. Additionally, any procedures involving use of an externally placed airway stent (eg T-tube) were excluded due to the potential for operative force during manipulation of the stent to bump the table and dislodge instruments from the holder.

Patients undergoing ELAM were randomized to have surgical instrument passes tracked with either the holder (device) or without it (control) for the duration of their operative case. Surgeons using the holder were instructed to verbalize their intention to retrieve from or put down an instrument into it. A custom software developed using MATLAB (The Math Works, Inc., Natick, Massachusetts) allowed in-person recorders (medical students and speech pathology students) to capture quantitative and qualitative data for each pass of a surgical instrument in both control and device cases. These variables included: (1) Instrument Pass Time (IPT)—the time from verbal request from the surgeon for an instrument to the time the tool was put in his or her hand (control) or retrieved from the holder (device); (2) Laterality of instrument pass; and (3) Errors associated with each pass (communication (eg wrong instrument) and/or inadvertent instrument drops). The laterality (or midline location) of a given laryngotracheal lesion within surgical field of view was also recorded for each case.

At the institution used for these tests, the ST is routinely located to the surgeon's right-hand side. Therefore, passes to and from the surgeon's left hand must occur over the patient laying on the OR table.

Post-operative qualitative metrics were collected from OR personnel using five-level Likert scales on a per case basis. Specifically, attending surgeons were queried on ST case-specific performance (1 [poor] to 5 [excellent]). ST's rated their familiarity with the current procedure/equipment for procedure (1 [never done before] to 5 [very familiar]) in every case and the ease of device setup (1 [poor] to 5 [very easy]), in holder cases. For each holder case, attending surgeons, otolaryngology residents, and ST's reported their satisfaction with (1 [not at all satisfied) to 5 [very satisfied]) and ease of device use (1 [poor] to 5 (very easy)].

Statistical analysis of the variables outlined above was performed with MATLAB. Outliers were defined as >3 standard deviations (SD) from the mean IPT. For purposes of analyzing ST performance and ST familiarity with procedure, scores were binned into low (1 to 3) or high (>3). Two sample T-tests were used to compare groups to each other. ANOVA tests were used if more than 2 groups were being compared.

### Example 2

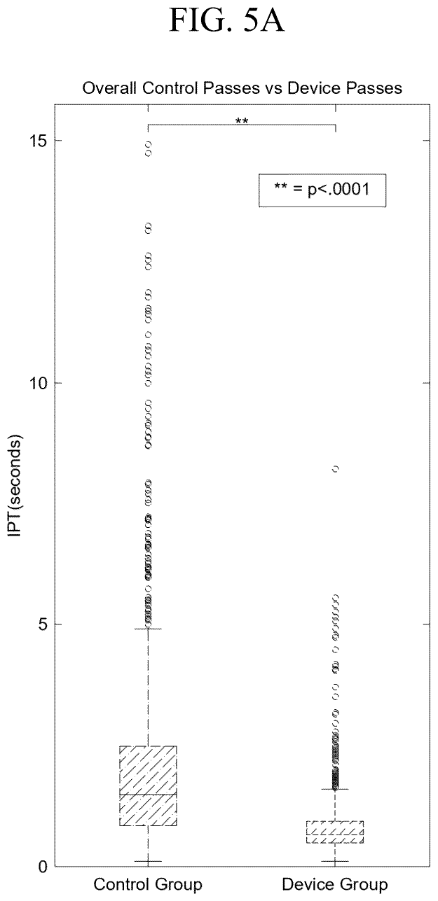

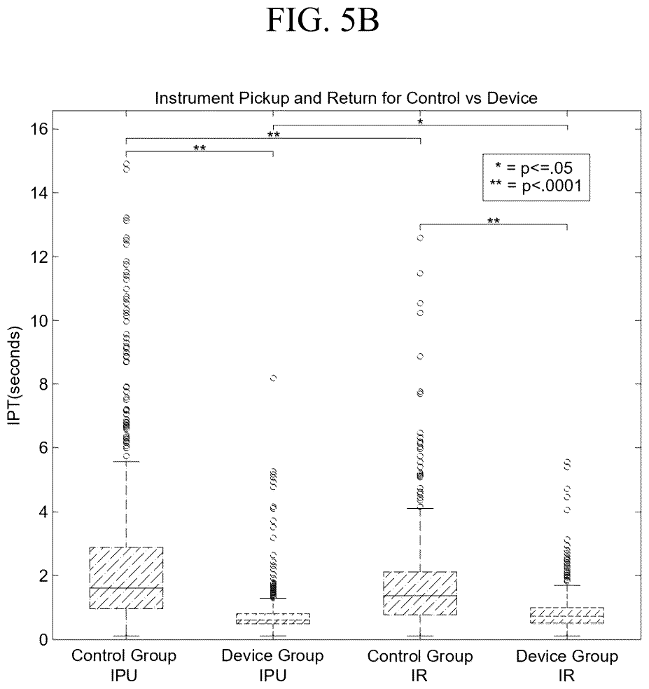

48 ELAM cases with close to 2400 instrument passes were analyzed as demonstrated in Table 1. Data was collected from three fellowship-trained laryngologists, 11 different otolaryngology residents, and 15 different ST's. Overall, 1208 instrument passes in control cases (n=23) were analyzed and compared to 1175 instrument passes in device cases (n=25). Mean overall IPT in control cases was 2.6 times higher than in device cases (Table 2). Additionally, as demonstrated in FIG. 5A, the variability of pass time was notably higher in non-device cases as evidenced by an inter-quartile range (IQR) of 1.65 seconds (control group) versus 0.44 seconds (device group). Similarly, the number and average magnitude of outliers was higher in the control group (n=94, IPT=7.81 s) than the device group (n=74, IPT=2.64 s).

Stratification of pass type by instrument pick up (IPU) or instrument return (IR) demonstrated similar statistically significant efficiency gains with the holder, as shown in FIG. 5B. There were also statistically significant differences within control and device groups. Returning instruments to the holder took longer than retrieving them, but the absolute mean difference was 0.08 seconds and both these times were still shorter than respective IPU and IR times without the holder. Conversely, mean IPU times were longer than IR times in control cases by 0.72 seconds.

The average number of passes per minute of operative time differed by operative pathology: 0.91 for phonosurgical, 0.81 for neoplastic, and 0.52 for airway stenosis. The average number of passes per case for each type of pathology was 54, 71, and 33 for phonosurgical, neoplastic and airway stenosis, respectively. Per Table 2, the IPT remained lower with the holder than without it, independent of operative pathology classification, laterality of lesion, or laterality of instrument pass. There were no statistically significant differences between left versus right-sided lesions or left versus right hand passes within either the control or device groups.

FIG. 6 demonstrates error rate during surgery with and without the holder. The holder did not increase instrument drop rates. In fact, it trended towards fewer instrument drops per case (1 drop every ˜6 cases) versus 1 drop every ˜4 cases without the holder, but this difference was not statistically significant. However, use of the holder did statistically significantly reduce communication errors by more than a factor of 7. Without the holder, there was almost one communication error between the surgeon and the ST in every case.

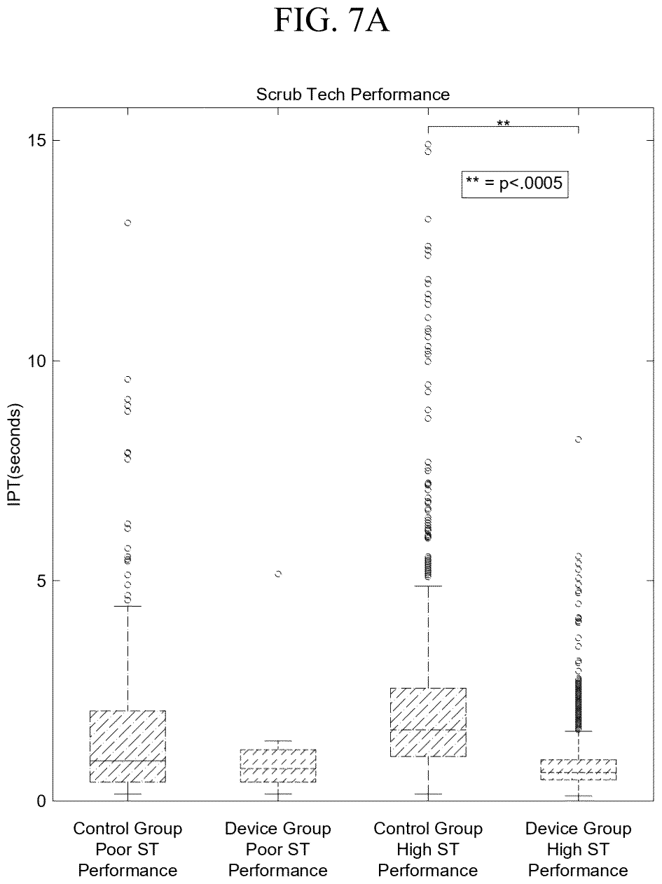

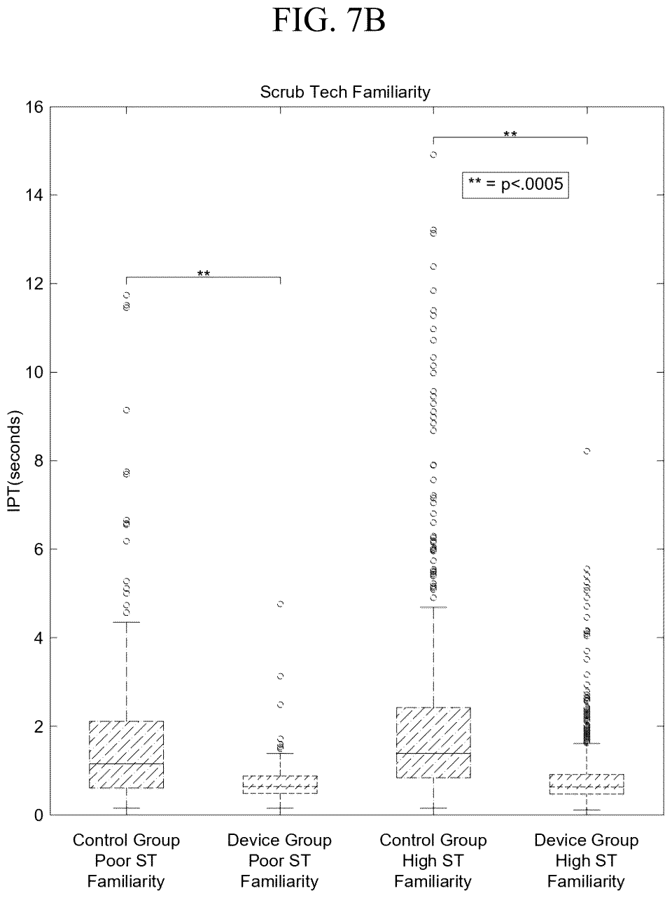

Attending surgeon-rated ST performance and self-rated ST familiarity with surgeon/procedure analysis is shown in FIGS. 7A and 7B. In general, poorer performing ST's did not show a significant reduction in IPT with the device, but higher performing ST's did. However, only 9 passes were recorded for poor-performing ST's using the holder. There were no significant differences in IPT between ST's with poor vs high procedure familiarity amongst either the control or holder groups. Qualitative assessments of the 25 holder-based cases are represented in Table 3.

### Tables

TABLE 1

| | Control | Device | Control vs |
|---|---|---|---|
| | Group | Group | Device p value |
| Total Number of Cases | 23 | 25 | |
| Total Number of Passes Recorded | 1208 | 1175 | |
| Laterality of Lesion | | | 0.98 |
| Left | 8 | 10 | |
| Right | 4 | 6 | |
| Left and Right | 2 | 2 | |
| Midline | 8 | 7 | |
| Type of Operative Pathology | | | 0.22 |
| Airway | 10 | 12 | |
| Neoplasm | 5 | 8 | |
| Phonosurgery | 7 | 5 | |

This table shows case demographics used to assess control vs device performance.

TABLE 2

| | | Control | Device | | |
|---|---|---|---|---|---|
| | Number | Group | Group | | Control |
| | of In- | Mean | Mean | Delta | vs |
| | strument | IPT | IPT | IPT | Device |
| | Passes | (seconds) | (seconds) | (seconds) | p-value |
| Overall | 2383 | 2.09 | 0.80 | 1.29 | <0.001 |
| Type of | | | | | |
| Operative | | | | | |
| Pathology | | | | | |
| Airway | 758 | 1.89 | 0.73 | 1.18 | <0.001 |
| Neoplasm | 973 | 2.28 | 0.79 | 1.49 | <0.001 |
| Phonosurgery | 652 | 2.02 | 0.85 | 1.16 | <0.001 |
| Laterality of | | | | | |
| Lesion | | | | | |
| Left | 397 | 2.29 | 0.81 | 1.48 | <0.001 |
| Right | 670 | 2.45 | 0.65 | 1.80 | <0.001 |
| Left and Right | 507 | 1.82 | 0.77 | 1.05 | <0.001 |
| Midline | 713 | 2.05 | 0.84 | 1.21 | <0.001 |
| Laterality of | | | | | |
| Instrument Pass | | | | | |
| Left hand pass | 689 | 2.07 | 0.84 | 1.23 | <0.001 |
| Right hand | 1694 | 2.09 | 0.78 | 1.31 | <0.001 |
| pass | | | | | |

Data stratified by type of operative pathology, lesion laterality, and pass laterality. Delta IPT represents difference in mean IPT for control group minus mean IPT for device group; n represents number of instrument passes

TABLE 3

| | Number of | Device Satisfaction | Device Ease of | |
|---|---|---|---|---|
| | Device | Rating | Use Rating | |
| | Cases | Mean (SD) | Mean (SD) | |
| Attending | 25 | 4.1 (0.93) | 4.1 | (0.86) |
| Resident | 20* | 3.7 (0.94) | 3.7 | (0.92) |
| Scrub-tech | 25 | 4.3 (0.62) | 3.9 | (1.1) |

Qualitative feedback on use of the holder device, rated on a 1 [low] to 5 [high] Likert scale. SD=standard deviation. *=five device cases were performed without a resident.

Having shown and described various embodiments of the present invention, further adaptations of the methods and systems described herein may be accomplished by appropriate modifications by one of ordinary skill in the art without departing from the scope of the present invention. For instance, the examples, embodiments, geometrics, materials, dimensions, and the like discussed above are illustrative and are not required. Accordingly, the scope of the present invention should be considered in terms of the following claims and is understood not to be limited to the details of structure and operation shown and described in the specification and drawings.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. An apparatus for securing surgical instruments to make them accessible by a surgeon while performing an operation on a patient, the apparatus comprising:
a. an articulated positioning arm comprising a first arm section and a second arm section; wherein the first arm section is oriented in a substantially horizontal position and the second arm section can be oriented in multiple positions;
b. an instrument support surface extending from the second arm section of the articulated positioning arm wherein the instrument support surface can be oriented in multiple positions;
c. a mounting interface that is capable of mounting the apparatus on a surgical bed; wherein the mounting interface is connected to the articulated positioning arm; and
d. a second device support connected to the mounting interface; wherein the second device support is configured to support one or more surgical tools.

2. The apparatus of claim 1 wherein the first arm section of the articulated positioning arm and the second arm section of the articulated positioning arm are connected at a perpendicular angle.

3. The apparatus of claim 1 wherein the instrument support surface comprises at least one removeable tool support.

4. The apparatus of claim 3 wherein the instrument support surface is configured to support endoscopic surgical tools.

5. The apparatus of claim 1 wherein the instrument support surface further comprises a surgical instrument tray, wherein the surgical instrument tray includes features that align and secure surgical instruments.

6. The apparatus of claim 1 wherein the instrument support surface is adjustable.

7. The apparatus of claim 1 wherein the instrument support surface can be positioned at an angle of from about 5 degrees to about 80 degrees from horizontal.

8. The apparatus of claim 1 wherein the instrument support surface can be positioned at an angle of from about 10 degrees to about 45 degrees from horizontal.

9. The apparatus of claim 1 wherein the second device support is configured to support one or more endoscopic surgical tools.

10. The apparatus of claim 1 wherein the second arm section of the articulated positioning arm comprises at least two ball joints.

11. The apparatus of claim 1 wherein the surgical instruments are endoscopic instruments and the second device support is an endoscopic tool holder.

12. The apparatus of claim 11 wherein the endoscopic instruments comprise microlaryngoscopic instruments.

13. The apparatus of claim 1 further comprising control surfaces that facilitate repositioning of the instrument support surface while maintaining sterility.

14. The apparatus of claim 1 wherein the instrument support surface further comprises flexible negative features that are capable of capturing distal portions of surgical instruments.

15. The apparatus of claim 1 wherein the instrument support surface further comprises features that extend from the surgical instrument tray which are capable of aligning surgical instruments and arresting any undesired lateral motion.

16. The apparatus of claim 1 wherein the instrument support surface further comprises one or more flexible protrusions that are capable of supporting the surgical instruments.

17. The apparatus of claim 1 wherein the apparatus is capable of being sterilized via autoclave.

18. The apparatus of claim 1 wherein the apparatus is capable of being sterilized using ethylene oxide.

19. The apparatus of claim 1 wherein the apparatus is capable of being sterilized via gamma radiation.

20. The apparatus of claim 1 wherein the surgical instruments are neurosurgical instruments.

21. The apparatus of claim 1 wherein the surgical instruments are ophthalmic instruments.

22. The apparatus of claim 1 wherein the surgical instruments are laparoscopic instruments.

23. The apparatus of claim 1 wherein the surgical instruments are otolaryngologic instruments.

24. The apparatus of claim 1 wherein the articulated positioning arm further comprises bend-and-stay arms, counterbalancing arms or combinations thereof.
